(19) United States
(12) Patent Application Publication
(10) Pub. No.: US 2004/0091921 A1
(43) Pub. Date: May 13, 2004

(54) Method for gene expression analysis and kit for gene expression analysis

(76) Inventors: Chihiro Uematsu, Kawasaki (JP); Kazunori Okano, Shiki (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/650,726

(22) Filed: Aug. 29, 2003

(30) Foreign Application Priority Data
Oct. 15, 2002 (JP) 2002-300790

(51) Int. Cl. C12Q 1/68; C12P 19/34

(52) U.S. Cl. 435/6; 435/91.2

(57) ABSTRACT

This invention provides a novel kit for gene expression analysis that can be universally used independent of the target nucleic acid sequence, and a simple method for gene expression analysis utilizing the same. This method comprises: subjecting a gene to be analyzed to real-time PCR detection using a primer comprising a base sequence specifically hybridizing to the target gene, a primer comprising a base sequence identical to the second base sequence, and the TaqMan® probe comprising a base sequence identical or complementary to the first base sequence, wherein the gene to be analyzed is prepared by introducing the first base sequence and the second base sequence, which are nonspecific to the base sequence of the target gene, into the target gene so that the second base sequence is bound to a position closer to the 5′ end than the first base sequence.

### TECHNICAL FIELD

The present invention relates to a method for gene expression analysis and a kit for gene expression analysis through fluorescent energy transfer. More particularly, the present invention relates to a method for gene expression analysis and a kit for gene expression analysis using a probe that can be universally used regardless of the target gene sequence, which is realized by the introduction of a sequence independent of the base sequence of the target gene therein.

### BACKGROUND ART

In the past, comparative RT-PCR was employed for DNA or RNA quantification. In comparative RT-PCR, a target gene-containing sample is first diluted to a variety of concentration levels to prepare standard samples, and the resulting standard samples are amplified to assay the amount of PCR products. Subsequently, the amount of PCR products are plotted based on the number of cycles to prepare a calibration curve, and the amount of the target gene at an unknown concentration level is determined based on the resulting calibration curve. Preparation of a calibration curve in comparative RT-PCR is, however, disadvantageously complicated since this procedure requires the determination of a region in which all the standard samples are exponentially amplified. In the assay of PCR products, fluorescent detection and electrophoresis are often combined. In general, the size of the amplification product is first confirmed by electrophoresis, the fluorescent intensity is assayed, and the amount of the amplification product must then be inspected.

Recently, several methods that enable real-time fluorescent detection without electrophoresis of the PCR product have been reported (U.S. Pat. No. 6,395,518, WO 97/42345, Nazarenko A. et al., Nucleic Acid Research, 1997, vol. 25, No. 12, pp. 2516-2521, Myakishev M. V. et al., Genome Research, 2001, Vol. 11, p163-169, Pamela M. et al., Proc. Natl. Acad. Sci., USA, August 1991, Vol. 88, pp. 7276-7280). In these methods, a probe that is devised to emit fluorescence by hybridizing to the PCR product through fluorescent energy transfer (for example, a TaqMan® probe or Molecular beacon) is used for real-time detection. During experiment, the PCR products can be assayed for each cycle, and thus, a region of PCR cycles in which the PCR products are exponentially amplified and a region of PCR cycles in which the PCR products have reached the plateau phase can be determined simply. Accordingly, labor in comparative RT-PCR was reduced, and this method rapidly became widely used as an effective comparative method for expressed genes.

In a method for detecting PCR products through fluorescent energy transfer, however, a probe or primer for detecting the target gene must be individually designed for each target gene. In addition, such a probe is disadvantageously cost-intensive due to using of fluorescent energy transfer and has difficulty that design guidelines are different from those of general primers.

In contrast, Whitcombe et al. have reported fluorescent assay for PCR products utilizing the TaqMan® probe that can be universally used regardless of the target gene sequence (Whitcombe D. et al., Clinical Chemistry, 1998, Vol. 44, No. 5, pp. 918-923). This assay targets genomic DNA and aims at SNP typing. In this method, a template-non-specific probe sequence and a Tag sequence are introduced into genomic DNA. This enables assay using the template-non-specific TaqMan® probe. In this method, however, two types of primer pairs are used, i.e., a primer pair for introduction and another primer pair that hybridizes to the Tag sequence of the synthesized DNA to amplify the DNA. This necessitates two different thermal cycles, and results in the unavoidable production of by-products. When real-time detection is carried out in one reaction vessel, accurate reactivity cannot be attained because of differences in Tm values between two types of probes. Accordingly, this assay is not convenient for the quantitative analysis of gene expression.

Objects of the present invention are to solve the problems of the prior art and to provide a novel kit for gene expression analysis that can be universally used regardless of the target gene sequence, as well as a simple method for gene expression analysis utilizing the same.

### DISCLOSURE OF THE INVENTION

In order to attain the above objects, the present invention provides the following method for gene expression analysis:

a method for gene expression analysis comprising:

subjecting a gene to be analyzed to nucleic acid amplification using a primer comprising a base sequence specifically hybridizing to a target gene, a primer comprising a base sequence identical to a second base sequence, a probe comprising a base sequence identical or complementary to a first base sequence, and labeled at one end with a fluorophore and at another end with a quencher, and thermostable DNA polymerase having 5′→3′ exonuclease activity,

digesting the probe hybridized to the first base sequence by the thermostable DNA polymerase at the time of the nucleic acid amplification, and

detecting a fluorescence emitted by the released fluorophore, thereby assaying the amount of the product of the nucleic acid amplification,

wherein the gene to be analyzed is prepared by introducing the first base sequence and the second base sequence, which are nonspecific to the base sequence of the target gene, into the target gene so that the second base sequence is bound to a position closer to the 5′ end than the first base sequence.

The first base sequence and the second base sequence are introduced into the target gene using a primer for introduction, which is constituted by 3 sequence portions. This primer for introduction comprises the first base sequence, which is closer to the 5′ end than the third base sequence comprising a sequence that specifically hybridizes to the target gene, and the second base sequence, which is closer to the 5′ end than the first base sequence. The first portion is a sequence identical or complementary to the probe for PCR detection, and the second portion is a sequence identical to the reverse primer for PCR detection. The first, second, and third portions may be continuous, or there may be a joining portion between two portions. Although the third sequence varies depending on the target gene sequence, the first and the second sequences can be freely designed independent of the target gene sequence.

In an embodiment of the present invention, the aforementioned primer for introduction comprising three sequence portions is first used as a reverse transcription primer, and first strand cDNA is synthesized from target RNA (mRNA), in the case of the mRNA expression analysis. This cDNA functions as a gene to be analyzed comprising the first base sequence and the second base sequence introduced into the target gene. Subsequently, the obtained gene to be analyzed (cDNA) is used as a template to perform real-time PCR detection using a forward primer, which hybridizes to a target gene at a portion closer to the 5′ end than the portion where the third portion of the primer for introduction hybridizes, a reverse primer consisting of a sequence identical to the second base sequence, and the TaqMan® probe consisting of a sequence identical or complementary to the first base sequence.

The TaqMan® probe is a DNA probe which is labeled at its 5′ end with a fluorophore and at its 3′ end with a substance that quenches the fluorescence emitted by the fluorophore through energy transfer (quencher). The TaqMan® probe does not emit fluorescence in a usual state because the 5′ end and the 3′ end are labeled with a fluorophore and a quencher, respectively. At the time of PCR, however, the probe hybridizes to the target sequence and elongation begins from the reverse primer. Hybridized probe was digested by 5′→3′ exonuclease activity of thermostable DNA polymerase, a free fluorophore is generated, and fluorescence is emitted.

In the method for gene expression analysis according to the present invention, nucleic acid amplification after reverse transcription is carried out in a single thermal cycle using a pair of primers. The term “cycle” refers to a process culminating in the synthesis of an elongated chain in general PCR. Specifically, the “cycle” comprises three steps of denaturing a double-stranded nucleic acid, annealing a primer and a probe with the gene to be analyzed, and elongation of the annealed primer. Thus, operation is simple, and there is no need to be concerned with the generation of by-products.

In the method for gene expression analysis according to the present invention, the use of two or more types of probes enables the simultaneous analyses of samples derived from several specimens using one vessel for one target gene. In such analysis, the Tm values of the aforementioned probes are preferably set at substantially the same levels to maintain the reactivity of each probe at consistent levels, thereby conducting accurate expression analysis.

The present invention also provides a kit for gene expression analysis. This kit comprises:

a probe comprising a sequence identical or complementary to a first base sequence, and labeled at one end with a fluorophore and at another end with a quencher, and

a primer comprising a sequence identical to a second base sequence and bound to a position closer to the 5′ end than the first base sequence,

wherein both the first base sequence and the second base sequence are nonspecific to a base sequence of a target gene and are introduced into the target gene.

When the simultaneous detection of several specimens is intended, the aforementioned kit preferably comprises two or more types of probes and the Tm values of those probes are substantially the same. Examples of two or more probes having substantially the same Tm values are probes which consist of several module sequences each consisting of 3 or 4 bases, where both terminal bases (base at the 5′ end and base at the 3′ end) of each module sequence are identical to each other, and each probe is constituted by rearranging module sequences having the identical terminal bases.

### BRIEF DESCRIPTION OF DRAWINGS

FIG. 1 is a diagram showing the procedures of real-time PCR detection using the universal probe of the present invention.

FIG. 2 is a diagram showing the sequence correlation among the universal probe of the present invention, the PCR reverse primer, and the reverse transcription primer.

FIG. 3 is a graph showing the results of detection by real-time PCR using the universal probe of the present invention and showing changes in the fluorescent signals relative to the PCR cycles. In the drawing, each dot forms serial 10-fold dilutions, the square plot indicates the highest initial concentration level of the target RNA, followed by a rhombic plot and a triangular plot, and a round plot indicates the lowest initial concentration level of the target RNA.

FIG. 4 is a graph comparing the results of detection by real-time PCR using the universal probe of the present invention and the results of real-time PCR by the Sunrise method, wherein a square plot represents a 100-fold dilution of a round plot, and a rhombic plot represents a further 100-fold dilution thereof.

FIG. 5 is a diagram schematically showing the sequences of two types of universal probes.

FIG. 6 is a diagram showing the procedures of detection by real-time PCR using two types of universal probes.

FIG. 7 is a graph showing the results of detection by real-time PCR using two types of universal probes when mouse liver-derived target RNA is used as a template (FIG. 7(a): probe A, FIG. 7(b): probe B).

FIG. 8 is a graph showing the results of detection by real-time PCR using two types of universal probes when mouse kidney-derived target RNA is used as a template (FIG. 8(a): probe A, FIG. 8(b): probe B).

FIG. 9 is a graph showing the results of detection by real-time PCR using two types of universal probes.

This description includes part or all of the contents as disclosed in the description of Japanese Patent Application No. 2002-300790, which is a priority document of the present application.

### PREFERRED EMBODIMENTS OF THE INVENTION

The present invention is hereafter described in detail with reference to the drawings.

### 1. Design of Primer for Introduction

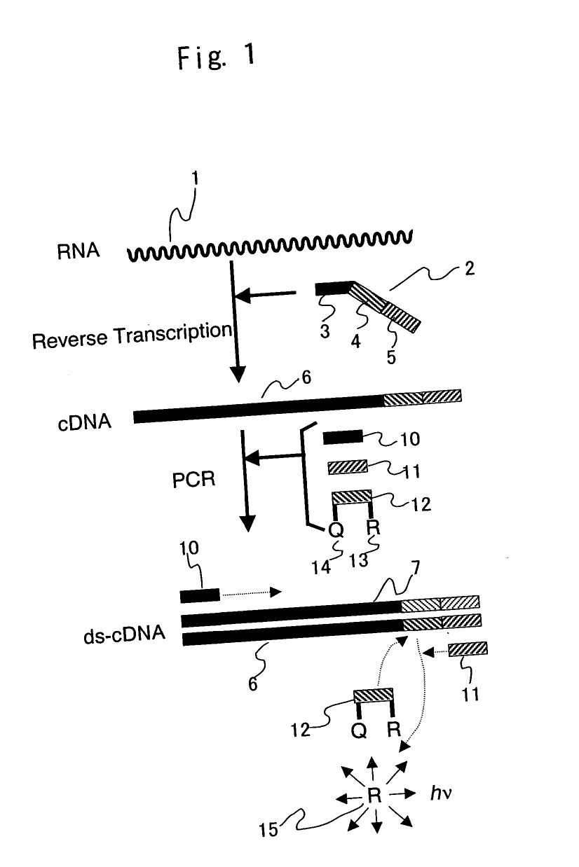

FIG. 1 is a diagram schematically showing the method for gene expression analysis according to the present invention, wherein the reference numeral 1 indicates a sample target RNA and the reference numeral 2 indicates a primer for introduction (reverse transcription). The primer 2 for introduction is constituted by the sequence portion 3 hybridizing to the target RNA, the sequence portion 4 located closer to the 5′ end than the sequence portion 3 and consisting of a sequence identical to the probe for PCR detection, and the sequence portion 5 located closer to the 5′ end than the sequence portion 4 and consisting of a sequence identical to the reverse primer 11 for PCR detection.

The aforementioned sequence portions 3, 4, and 5 may be continuous, or there may be a joining portion between two portions. The length of the sequence portion 3 is not particularly limited, and it is preferably about 18 to 25 bases. The length of the sequence portion 4 is not particularly limited, and it is preferably about 18 to 30 bases. The length of the sequence portion 5 is not particularly limited, and it is preferably about 18 to 25 bases. Although the sequence portion 3 varies depending on the target gene sequence, the sequence portions 4 and 5 can be freely designed independent of the target gene sequence.

### 2. Synthesis of the Gene to be Analyzed (Reverse Transcription into cDNA)

The aforementioned primer for introduction is used as a reverse transcription primer, and cDNA is synthesized from mRNA containing the target gene. Reverse transcription is carried out in accordance with a method known in the art. The aforementioned primer, the reverse transcriptase, and a substrate are added to a template RNA-containing reaction solution, and the mixture may be incubated at 35° C. to 45° C. for about 30 to 60 minutes. As a result of this reverse transcription, cDNA comprising the first base sequence 4 and the second base sequence 5, which are non-specific to the base sequence of the target gene and are introduced into the target gene, is synthesized as the gene to be analyzed.

### 3. Design of PCR Primer and Universal Probe

The design of a PCR primer for amplifying the gene to be analyzed (cDNA) synthesized above and a universal probe for detecting this gene are described.

As shown in FIG. 1, the forward primer 10, which hybridizes to the gene to be analyzed, and the reverse primer 11, which has a sequence identical to the sequence portion 5 introduced into the gene to be analyzed, are used as the aforementioned PCR primers.

The probe 12, which has a sequence identical to the sequence portion 4 introduced into the gene to be analyzed, is used as a probe for detecting PCR products. The probe 12 is labeled with a fluorophore 13 indicated as “R” in the drawing and a quencher 14 indicated as “Q” in the drawing. In an intact state, fluorescence of the fluorophore 13 is eliminated through fluorescent energy transfer. However, the probe 12 releases the fluorophore and allows it to emit light with the progress of PCR. Examples of the fluorophore that can be used include coumarin, fluorescein, tetrachlorofluorescein, hexachlorofluorescein, Lucifer yellow, rhodamine, BODIPY, tetramethylrhodamine, Cy3, Cy5, Cy7, eosine, Texas Red, ROX, FAM, and VIC. Examples of quenchers include 4-(4′-dimethylaminophenylazo)benzoic acid (DABCYL), DABMI, BHQ-0, BHQ-1, BHQ-2, and methyl red. Among the aforementioned fluorophores, those that can quench fluorescence when it is brought into contact with another type of fluorophore can be used as a quencher.

The probe 12 should be designed to have a Tm value of 5° C. to 15° C., preferably about 10° C. higher than that of the forward primer 10 or reverse primer 11, so that this probe can hybridize to the target sequence more quickly than the forward primer 10 or reverse primer 11. This Tm value is adjusted using, for example, a substance that enhances the Tm value of the probe, i.e., a Tm enhancer. An example of the Tm enhancer that can be used is a DNA-binding substance such as Minor Groove Binder (MGB). The 3′ end of the probe 12 should be phosphorylated in order to prevent it from being elongated by thermostable DNA polymerase.

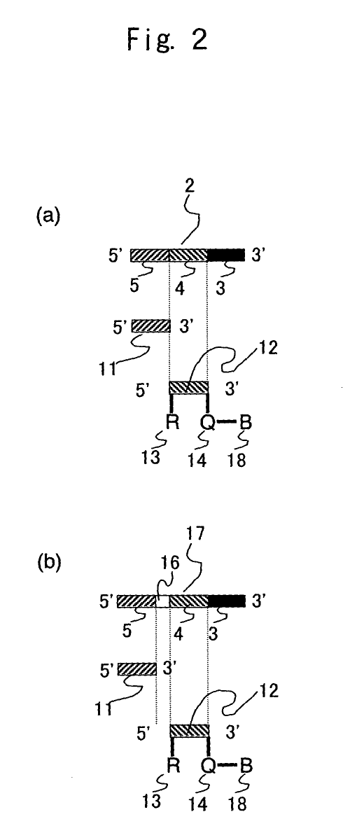

The sequence correlation among the reverse transcription primer 2, the reverse primer 11, and the probe 12 is described in more detail with reference to FIG. 2. In this drawing, the 5′ ends of the probe and the primer are on the left side and the 3′ ends thereof are on the right side.

As shown in FIG. 2(a), the reverse transcription primer 2 is constituted by the sequence portion 3 hybridizing to target RNA, the sequence portion 4 consisting of a sequence identical to the probe 12, and the sequence portion 5 consisting of a sequence identical to the reverse primer 11. The sequence portion 5 is located closest to the 5′ end, the sequence portion 4 is located closer to the 3′ end than the sequence portion 5, and the sequence portion 3 is located closer to the 3′ end than the sequence portion 4. The probe 12 is labeled at its 5′ end with the fluorophore 13 and at its 3′ end with the quencher 14. The Tm enhancer 18 is bound to the quencher 14.

As shown in FIG. 2(b), there may exist a sequence portion 16, which is a joining portion between two sequence portions, in addition to the sequence portion 3 at which the reverse transcription primer 17 hybridizes to target RNA, the sequence portion 4 consisting of a sequence identical to the probe 12, and the sequence portion 5 consisting of a sequence identical to the reverse primer 11. The length of the joining portion is not particularly limited, and it is preferably about 1 to 5 bases.

The probe 12 and the reverse primer 11 should be in a physical relationship such that, when they hybridize to a DNA strand, the probe 12 is hydrolyzed upon the elongation of the reverse primer 11 by DNA polymerase to allow the probe 12 to emit fluorescence. The sequence portion 4 consisting of a sequence identical to the probe 12 may be located closer to the 3′ end than the sequence portion 5 consisting of a sequence identical to the reverse primer 11. The 3′ end of the sequence portion 5 may be in contact with the 5′ end of the sequence portion 4 as shown in FIG. 2(a). Alternatively, other base sequence may be present therebetween as shown in FIG. 2(b).

The sequence of the probe 12 can be designed independently of the sequence of target RNA. When assaying another target RNA, redesign of the sequence portion 3 of the reverse transcription primer 2 and the sequence of the forward primer 10 is sufficient, and the probe 12 can be used as a universal probe. Also, the reverse primer 11 can also be used as a common primer.

### 4. PCR and Fluorescent Emission

PCR is carried out in accordance with a method known in the art by putting the gene to be analyzed (cDNA), the forward primer, the reverse primer, and the probe in a reaction tube and using thermostable DNA polymerase having 5′→3′ exonuclease activity.

Processes for PCR and fluorescent emission are described with reference to FIG. 1. In the early stage of the aforementioned PCR, the forward primer 10 hybridizes to the first strand cDNA 6 and elongated. Thus, second strand cDNA 7 is synthesized. The second strand cDNA 7 has a sequence complementary to the reverse primer 11 and a sequence complementary to the probe 12. The Tm value of the probe 12 is enhanced by the Tm enhancer (DNA-binding substance) that is binding to the quencher 14, and thus, the probe 12 can hybridize to the target sequence more quickly than a primer. Accordingly, the probe 12 preferentially hybridizes to the second strand cDNA 7.

Further, the forward primer 10 hybridizes to the first strand cDNA 6, the reverse primer 11 hybridizes to the second strand cDNA 7, and these primers are elongated. Upon the elongation of the reverse primer 11, the probe 12 which hybridizes to the second strand cDNA 7 is hydrolyzed from the 5′ end by 5′→3′ exonuclease activity of DNA polymerase. As a result of this hydrolysis, the fluorophore 13 is released from the probe 12, the free fluorophore 15 is generated, and fluorescence is emitted.

FIG. 1 shows a case where the probe 12 is identical to the sequence portion 4 of the reverse transcription primer 2. Detection can be similarly carried out when the probe 12 is complementary to the sequence portion 4. In such a case, the probe 12 hybridizes to the first strand cDNA 6 and hydrolyzed upon the elongation of the forward primer 10.

As described above, the probe 12 hybridizes to the first strand cDNA 6 or the second strand cDNA 7 that is generated during PCR, is hydrolyzed upon the elongation of the forward primer 10 or the reverse primer 11, and emits fluorescence. Accordingly, the amount of emitted fluorescence is increased depending on the amount of cDNA amplified by PCR. Thus, the amount of the target RNA contained in the sample can be measured. In this case, by-products can be reduced because of a single thermal cycle of PCR, unlike the example described in Whitcombe et al. (Whitcombe D. et al., Clinical Chemistry, 1998, Vol. 44, No. 5, pp. 918-923). Thus, accuracy of the quantitative analysis of target RNA can be improved.

### 5. Simultaneous Analyses of Several Specimens

A method for simultaneously analyzing samples derived from several specimens (simultaneous analyses of several specimens) using two or more types of universal probes and using one vessel for one target gene is described.

### 5.1 Design of Two or More Types of Universal Probes

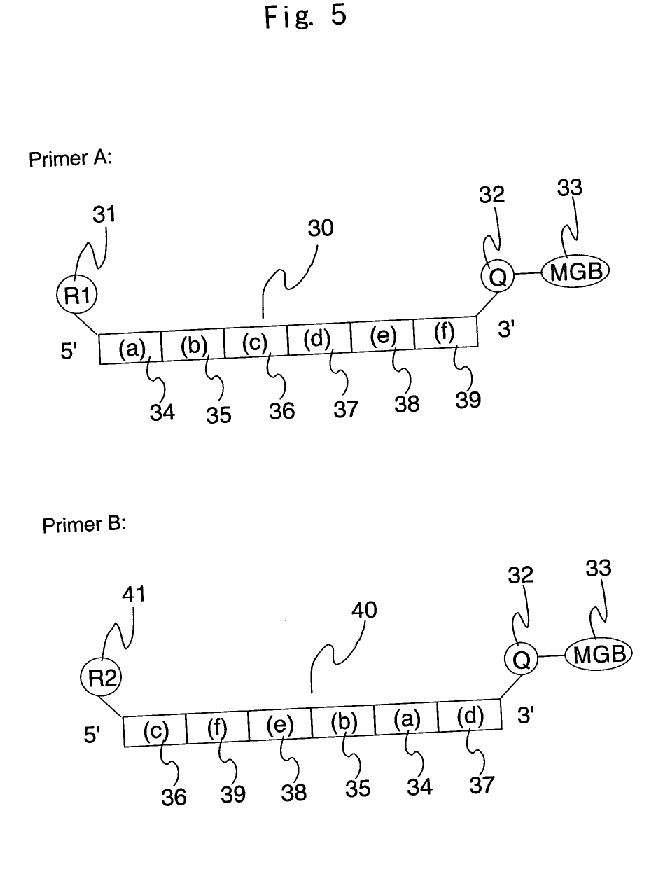

FIG. 5 schematically shows structures of two types of universal probes, probe A (30 in FIG. 5) and probe B (40 in FIG. 5), used when simultaneously analyzing several specimens.

Probe A is labeled at its 5′ end with a fluorophore 31 indicated as “R1” in the drawing and at its 3′ end with a quencher 32, which quenches the R1-derived fluorescence through fluorescent energy transfer. As with the case of probe A, probe B is labeled at its 5′ end with the fluorophore 41 indicated as “R2” in the drawing and at its 3′ end with the quencher 32 for quenching fluorescence. If necessary, the Tm enhancer 33, which binds to the minor groove of DNA, may be linked to the quencher 32 of each probe.

R2 that labels the 5′ end of probe B should be a fluorophore that emits light at a fluorescent wavelength different from that of R1. Thus, it can be determined whether or not the fluorescence is derived from probe A or probe B based on the difference in fluorescent wavelengths between R1 and R2.

Further, probe A and probe B should be designed to hybridize to each target gene with the same reaction properties. As shown in FIG. 5, the sequence of probe A and that of probe B are constituted by module sequences 34 to 39 consisting of 3 or 4 bases. The number of module sequences that constitute the probe is not particularly limited. In general, it is preferably about 5 to 8. Both terminal bases of each module sequence are the same. The sequence of probe B is constituted by rearranged the order of modules having terminal bases identical as compared with the sequence of probe A. Since modules having the same terminal bases are rearranged, the base sequences at the joining portions between modules of probe A are the same as those of probe B. Probe A and probe B are constituted by the same modules. Thus, the thermodynamic properties of probe A are equivalent to those of probe B, and Tm values of probe A and probe B are identical based on the calculation by the nearest neighbor method. Specifically, entire sequence of probe A is different from entire sequence of probe B. However, these probes substantially have the same Tm values, and can hybridize to their complementary sequences with the same reaction properties even though they were allowed to simultaneously react in the same reaction tube. Thus, accurate analysis can be made when these probes are used for quantitative analysis. The design of two types of probes is described above. Three or more types of probes can be similarly designed.

### 5.2 Synthesis of the Gene to be Analyzed (Reverse Transcription into cDNA)

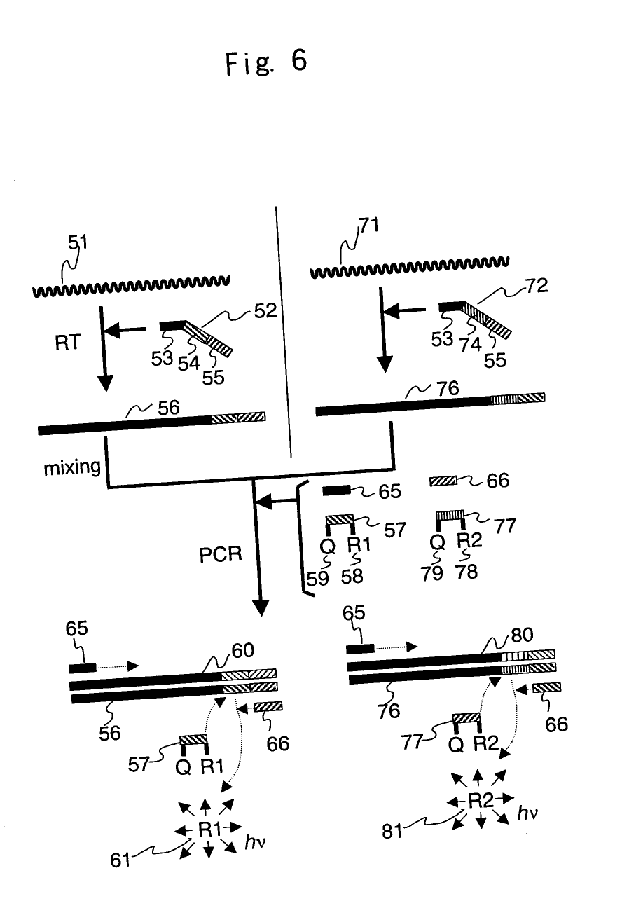

FIG. 6 schematically shows a method for analyzing the expression of genes derived from each sample using probe A and probe B to amplify the target genes derived from two types of samples (samples 1 and 2) by PCR in a reaction tube. First, cDNA is prepared from RNA extracted from sample 1. The numerical reference 51 indicates RNA extracted from sample 1, and the numerical reference 52 indicates a reverse transcription primer. The reverse transcription primer 52 is constituted by the sequence portion 53 hybridizing to the target RNA, the sequence portion 54 located closer to the 5′ end than the sequence portion 53 and consisting of a sequence identical to the probe for PCR detection, and the sequence portion 55 located closer to the 5′ end than the sequence portion 54 and consisting of a sequence identical to the reverse primer 66 for PCR detection. Reverse transcription is carried out using this primer in the same manner as in 4. above. Thus, the first strand cDNA 56 in which the sequence portion 54 and the sequence portion 55 have been introduced is obtained.

Another cDNA is synthesized from RNA extracted from sample 2 in a tube different from the reaction tube in which the first strand cDNA 56 was prepared. The numerical reference 71 indicates RNA extracted from sample 2, and the numerical reference 72 indicates a reverse transcription primer. The reverse transcription primer 72 is constituted by the sequence portion 53 hybridizing to the target RNA, the sequence portion 74 located closer to the 5′ end than the sequence portion 53 and consisting of a sequence identical to the probe for PCR detection, and the sequence portion 55 located closer to the 5′ end than the sequence portion 74 and consisting of a sequence identical to the reverse primer 66 for PCR detection. The sample 2 is similarly subjected to reverse transcription to obtain the first strand cDNA 76 comprising the sequence portion 74 and the sequence portion 55 introduced therein. The sequence portion 53 of the reverse transcription primer 52 and the sequence portion 53 of the reverse transcription primer 72 hybridize to the same target RNA, and thus, these sequences are identical to each other. Also, the sequence portion 55 of the reverse transcription primer 52 is identical to the sequence portion 55 of the reverse transcription primer 72. The sequence portion 54 of the reverse transcription primer 52 and the sequence portion 74 of the reverse transcription primer 72 correspond to probe A and probe B as shown in FIG. 5, respectively, and they have the same Tm values.

As described in 3. above, each probe should be designed to be hybridizable with the target sequence more quickly than a primer. Specifically, the probe 57 and the probe 77 are designed to have the Tm values of 5° C. to 15° C., preferably about 10° C. higher than those of the forward primer 65 or the reverse primer 66. This Tm value is adjusted using, for example, a substance for enhancing the Tm value of the probe, i.e., a Tm enhancer.

### 5.3 Competitive PCR

Competitive PCR is schematically described with reference to FIG. 6. A part of the resulting first strand cDNA 56 and a part of the first strand cDNA 76 are taken out of the reaction tube, and equivalent amounts thereof are mixed in a new reaction tube. The resultant is designated as a PCR template. The forward primer 65, which hybridizes to the first strand cDNA 56 and the first strand cDNA 76, and the reverse primer 66, which has a sequence identical to the sequence portion 55 introduced into the first strand cDNA 56 and the first strand cDNA 76, are used as the PCR primers.

The probe 57, which has the same sequence as the sequence portion 54 introduced into the first strand cDNA 56, and the probe 77, which has the same sequence as the sequence portion 74 introduced into the first strand cDNA 76, are used as universal probes for detecting PCR products.

The probe 57 is labeled with the fluorophore 58 indicated as “R1” in the drawing and the quencher 59 indicated as “Q” in the drawing. The probe 77 is labeled with the fluorophore 78 indicated as “R2” in the drawing and the quencher 79 indicated as “Q” in the drawing. The quenchers 59 and 79 may be the same if such a quencher can eliminate fluorescence emitted by the fluorophores 58 and 78, respectively. When the probe 57 and the probe 77 are in an intact state, fluorescence of the fluorophore 58 and the fluorophore 78 are eliminated through fluorescent energy transfer.

Amplification by PCR is carried out using thermostable DNA polymerase having 5′→3′ exonuclease activity. In the early stage of PCR, the forward primer 65 hybridizes to the first strand cDNA 56 and elongated. Thus, second strand cDNA 60 is synthesized. Also, the forward primer 65 hybridizes to the first strand cDNA 76 and elongated. Thus, the second strand cDNA 80 is synthesized. The second strand cDNA 60 has a sequence complementary to the reverse primer 66 and a sequence complementary to the probe 57. The second strand cDNA 80 has a sequence complementary to the reverse primer 66 and a sequence complementary to the probe 77.

With the progress of PCR, the second strand cDNA 60 and the second strand cDNA 80 are amplified from their own templates, respectively. The probe 57 hybridizes to the second strand cDNA 60, and the probe 77 hybridizes to the second strand cDNA 80. Upon the elongation of the reverse primer 66 hybridizing to the second strand cDNA 60, the probe 57 hybridizing to the second strand cDNA 60 is hydrolyzed from the 5′ end by 5′→3′ exonuclease activity of DNA polymerase. As a result of the hydrolysis, the fluorophore 58 is released from the probe 57, a free fluorophore 61 is generated, and fluorescence is emitted. Upon the elongation of the reverse primer 66 hybridizing to the second strand cDNA 80, the probe 77 hybridizing to the second strand cDNA 80 is hydrolyzed from the 5′ end by 5′→3′ exonuclease activity of DNA polymerase. As a result of the hydrolysis, the fluorophore 78 is released from the probe 77, a free fluorophore 81 is generated, and fluorescence is emitted.

The ratio between the free fluorophore 61 and free fluorophore 81 that are generated by PCR depends on the quantitative ratio between the cDNA 56 and cDNA 76 that are present in the reaction tube before the PCR. Thus, comparison of the intensities of fluorescent signals emitted from these two fluorophores enables the assay of the abundance ratio between the cDNA 56 and cDNA 76. Specifically, the expression levels of one gene in several samples can be simply compared.

### 6. Kit

As described above, the first base sequence and the second base sequence are introduced into a target gene, which are unrelated to base sequences of target genes. This can provide the TaqMan® probe (a universal probe) that can be universally used for any kind of target gene. When detecting another target gene, it is sufficient to modify only the sequence of the portion which hybridizes to the target gene of the primer for introduction (primer for reverse transcription) and the sequence of the forward primer. In addition to the aforementioned probe, the reverse primer for real-time PCR can be universally used for any target genes since this primer is independent of the base sequence of the target gene.

More specifically, the present invention provides a kit for gene expression analysis comprising a probe and a primer that can be used universally. This kit may be used for the analysis of a single specimen, or it may be used for the simultaneous analyses of several specimens. The characteristics and the constructions of the essential elements of this kit, the universal probe and the reverse primer, are as described above.

The kit according to the present invention may comprise other reagents or the like that are necessary for gene expression analysis in addition to the universal probe and the primer as the essential elements. Examples thereof include a Tm regulator such as betain or trimethylamine-N-oxide, a buffer that imparts suitable conditions for the enzyme reaction, and other reagents that are necessary for detecting synthesis products. This kit may supply a reagent, which is necessary for one reaction, in a fractionated state in a reaction vessel.

### EXAMPLES

The present invention is hereafter described in more detail with reference to the following examples, although it is not limited to these examples.

### Example 1

### Gene Expression Analysis of GAPDH Using a Universal Probe

### 1. GAPDH Expression Analysis Using a Universal Probe

### (1) Method of Examination

Reverse transcription was performed using RNA extracted from a mouse liver as a sample and using the following reverse transcription primer for glyceraldehyde-3-phosphate dehydrogenase (GAPDH) to prepare first strand cDNA. Reverse transcription was performed using SuperScript II reverse transcriptase (Invitrogen).

Reverse Transcription Primer: 5′-TAC GAC TCA CTC TAC GCC CA CCC TTCT CAC TGTT CTC TCAT GGA TGC AGG GAT GAT GTT C-3′ (SEQ ID NO: 1)

In the reverse transcription, 2 pmol of reverse transcription primer, the total RNA extracted from a mouse liver, and SuperScript II reverse transcriptase were mixed in a reaction buffer, and the mixture was incubated at 42° C. for 50 minutes. Thereafter, ribonuclease H was added to the reaction solution and the resultant was incubated at 37° C. for 20 minutes in order to digest the RNA remaining in the reaction solution.

PCR products were detected using the Sequence Detection system 7900 (Applied BioSystems). The TaqMan® Universal PCR Master Mix kit (Applied BioSystems) as thermostable DNA polymerase and a substrate were used. PCR was carried out under the following conditions. The first strand cDNA synthesized above, the following forward primer (15 pmol), and the following reverse primer (15 pmol) were mixed with 5 pmol of probe labeled at its 5′ end with a fluorophore FAM and at its 3′ end with a quencher NFQ and a Tm enhancer MGB in 20 μl of reaction solution. The mixture was incubated at 95° C. for 10 minutes and a thermal cycle of 95° C. for 15 seconds and 60° C. for 1 minute was repeated 50 times.

| | | |
|---|---|---|
| Forward Primer: | 5′-TGC ACC ACC AAC TGC TTA G-3′ | (SEQ ID NO:2) |
| Reverse primer: | 5′-TAC GAC TCA CTC TAC GCC GA-3′ | (SEQ ID NO:3) |
| Probe: | 5′-(FAM)-CCC TTCT CAC TGTT CTC TCAT-(NFQ)-(MGB)-3′ | (SEQ ID NO:4) |

### (2) Results

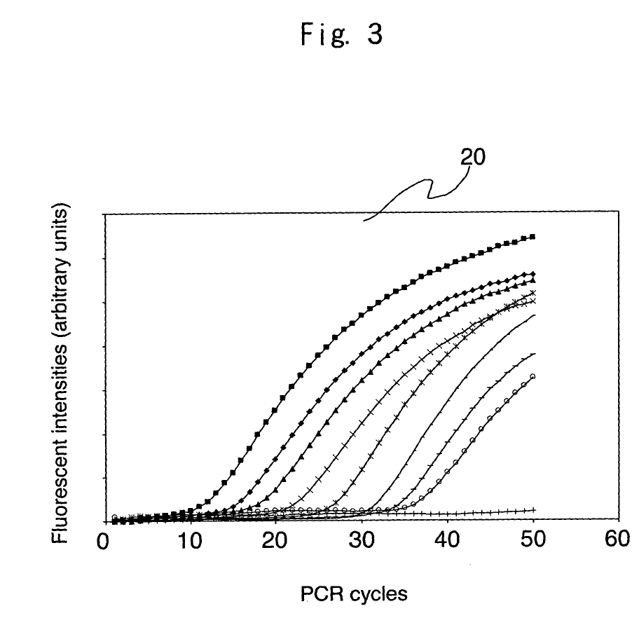

FIG. 3 shows the results attained by fluorescent detection for each PCR cycle. The horizontal axis of the graph indicates PCR cycles, and the vertical axis indicates the fluorescent intensity (arbitrary units). The graph shows changes in fluorescent signals from samples containing target RNAs at various concentration levels. A square plot indicates the highest initial concentration level of the target RNA, followed by a rhombic plot and a triangular plot, and a round plot indicates the lowest initial concentration level of the target RNA. Each plot forms serial 10-fold dilutions. In the real-time detection of PCR products, a cycle in which the intensity of a fluorescent signal derived from PCR products exceeds the threshold is defined as a threshold cycle (C_{T}), and this is used as an indicator for the initial concentration level of the target RNA. In FIG. 3, an increase in the intensity of a fluorescent signal was detected depending on the initial concentration level of the target RNA in the concentration range of at least 10⁸. The results indicate that the C_{T} cycle was analyzed consistently with the initial concentration level. In the graph in FIG. 3, “+” plots indicate changes in the fluorescent signals derived from a sample containing no target RNA.

### 2. Comparison with the Sunrise Method

### (1) Method of Examination

A conventional method for expression analysis using the Sunrise primer was compared to the method of the present invention. At the outset, target RNAs at various concentration levels were subjected to PCR in the same manner as in 1. above to assay changes in fluorescent signals. PCR was then carried out using the same PCR template used in the present invention, 20 pmol of the forward primer having a base sequence identical to SEQ ID NO: 2, and 20 pmol of the following reverse Sunrise primer labeled at its 5′ end with a fluorophore FAM and with its thymine at the twenty third base aminated and labeled with a quencher (DABCYL).

| | | |
|---|---|---|
| Forward Primer: | 5′-TGC ACC ACC AAC TGC TTA G-3′ | (SEQ ID NO:2) |
| Reverse Sunrise | 5′-(FAM)-AGCG ATGC ACCCTCA GCAT CGCT* CCC TTCT CAC TGTT CTC TCAT-3′ | (SEQ ID NO:5) |
| Primer: | | |

The above “(FAM)” indicates a fluorophore FAM, and “*” indicates a quencher (DABCYL). PCR and assay conditions were the same as those used in 1.

### (2) Results

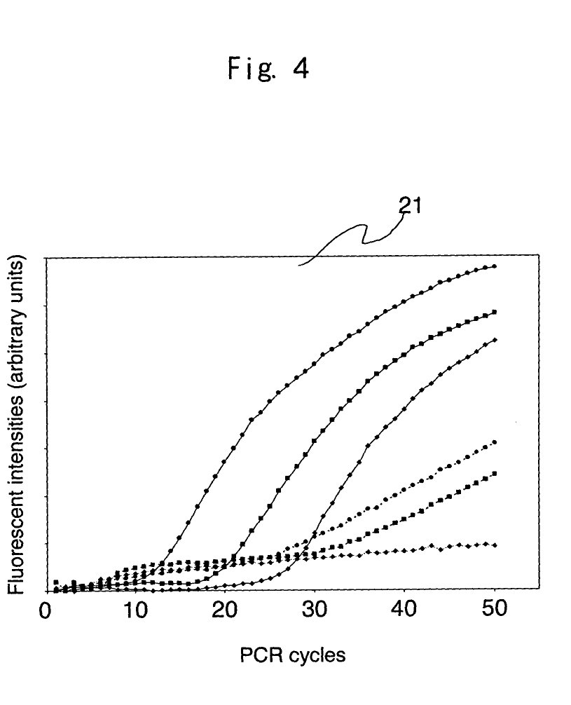

FIG. 4 shows the results of comparison of changes in the fluorescent signal intensity attained by the Sunrise method and that attained by the method of the present invention. In the drawing, a solid line represents changes in the fluorescent signal when detecting GAPDH derived from a mouse liver by the method of the present invention. A dashed line represents changes in the fluorescent signal when detecting by the Sunrise method. The horizontal axis indicates PCR cycles, and the vertical axis indicates the fluorescent intensity (arbitrary units). A square plot represents a 100-fold dilution of a round plot, and a rhombic plot represents a further 100-fold dilution thereof.

In the cases where there was no dilution and cases of serial 100-fold dilutions, fluorescent signal values were both increased by the method of the present invention and the Sunrise method. In the case of the Sunrise method, however, PCR cycles at which the fluorescent signal value was increased was at least 10 cycles later than the case of method of the present invention, and the fluorescent signal value was small. In the serial 100²-fold dilutions, while the fluorescent signal value was increased by the method of the present invention, the fluorescent signal value was not changed very much in the case of the Sunrise method. Accordingly, the detection sensitivity of the method of the present invention was higher than that of the Sunrise method.

### Example 2

### Simultaneous Assay of Several Specimens

An example is presented in which target genes derived from several samples were amplified by PCR in one reaction tube and detected according to the method of the present invention.

### (1) Method of Examination

Reverse transcription was carried out in the same manner as in Example 1 using RNA extracted from a mouse liver and RNA extracted from a mouse kidney as templates. The first strand cDNA of each GAPDH was prepared. The reverse transcription primer for RNA derived from a mouse liver was the same as that used in Example 1, and the following primer (SEQ ID NO: 6) was used as the reverse transcription primer for RNA derived from a mouse kidney.

The reverse transcription primer for RNA derived from a mouse liver and that for RNA derived from a mouse kidney were designed in accordance with FIG. 6. Specifically, a sequence portion 53 hybridizing to target RNA and the sequence portion 55 consisting of a sequence identical to the reverse primer 66 for PCR detection existed in common. The sequence portion 54 and the sequence portion 74 (underlined portions) consisting of a sequence identical to the probe for PCR detection located therebetween were different.

| | | |
|---|---|---|
| Reverse Transcription Primer (Liver): | 5′-TAC GAC TCA CTC TAC GCC CA CCC TTCT | (SEQ ID NO:1) |
| | CAC TGTT CTC TCAT GGA TGC AGG GAT GAT GTT C-3′ | |
| Reverse Transcription Primer (Kidney): | 5′-TAC GAC TCA CTC TAC GCC CA CAC TCAT | (SEQ ID NO:6) |
| | CTC TTCT CCC TGTT GGA TGC AGG GAT GAT GTT C-3′ | |

PCR was performed by taking a part of cDNA derived from each obtained sample out of the reaction tube, mixing equivalent amounts thereof in a new reaction tube, using the resultant as a template, and using the primers (SEQ ID NOs: 2 and 3) used in Example 1 and two types of probes.

The probe for detecting liver-derived GAPDH (probe A) was the same probe as that used in Example 1 (SEQ ID NO: 4), and the probe for detecting kidney-derived GAPDH (probe B) was the probe as shown below (SEQ ID NO: 7).

| | | |
|---|---|---|
| Probe A: | 5′-(FAM)-CCC TTCT CAC TGTT CTC TCAT-(NFQ)-(MGB)-3′ | (SEQ ID NO:4) |
| Probe B: | 5′-(VIC)-CAC TCAT CTC TTCT CCC TGTT-(NFQ)-(MGB)-3′ | (SEQ ID NO:7) |

Both probes are designed to have substantially the same Tm in accordance with FIG. 5 and FIG. 6. Probe A is labeled at its 5′ end with a fluorophore FAM, probe B is labeled at its 5′ end with a fluorophore VIC, and both probes are labeled at their 3′ ends with quenchers, respectively. A Tm enhancer MGB that binds to a minor groove of DNA is linked to each quencher. PCR and assay conditions were the same as those used in 1.

### (2) Results

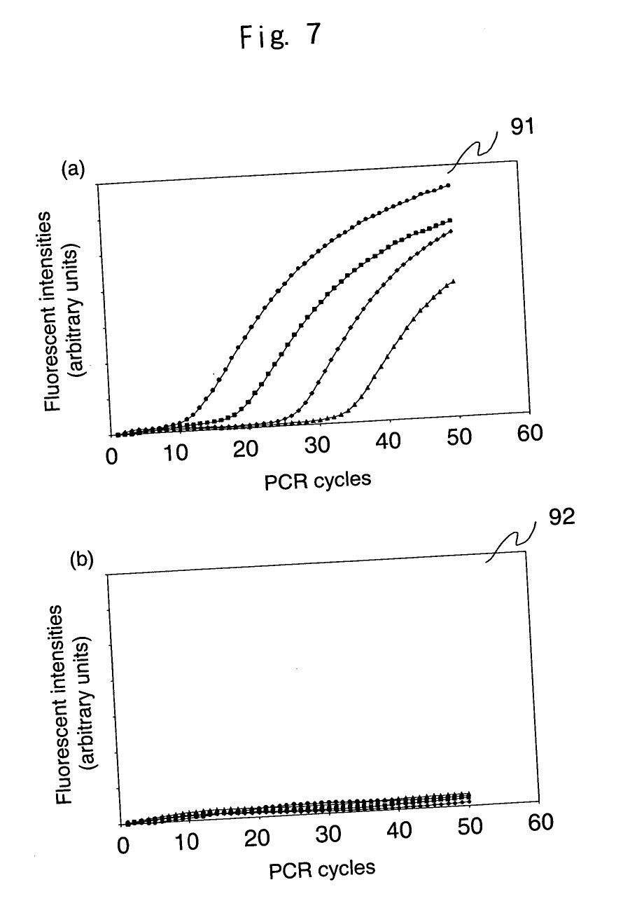

FIG. 7 shows the results of experiments in which mouse liver-derived target RNA was used as a template at various concentration levels and two types of probes were used for detection. The horizontal axis of the graph represents PCR cycles, and the vertical axis represents relative fluorescent intensities (arbitrary units) of a fluorophore. FIG. 7(a) is a graph showing changes in the intensity of the fluorescent signal when detecting PCR products of a mouse liver-derived target RNA using probe A, and FIG. 7(b) is a graph showing that using probe B. As is apparent from FIG. 7(b), a sample did not contain any mouse kidney-derived target RNA at all, and thus, a fluorescent signal derived from probe B was not substantially amplified in the PCR cycle.

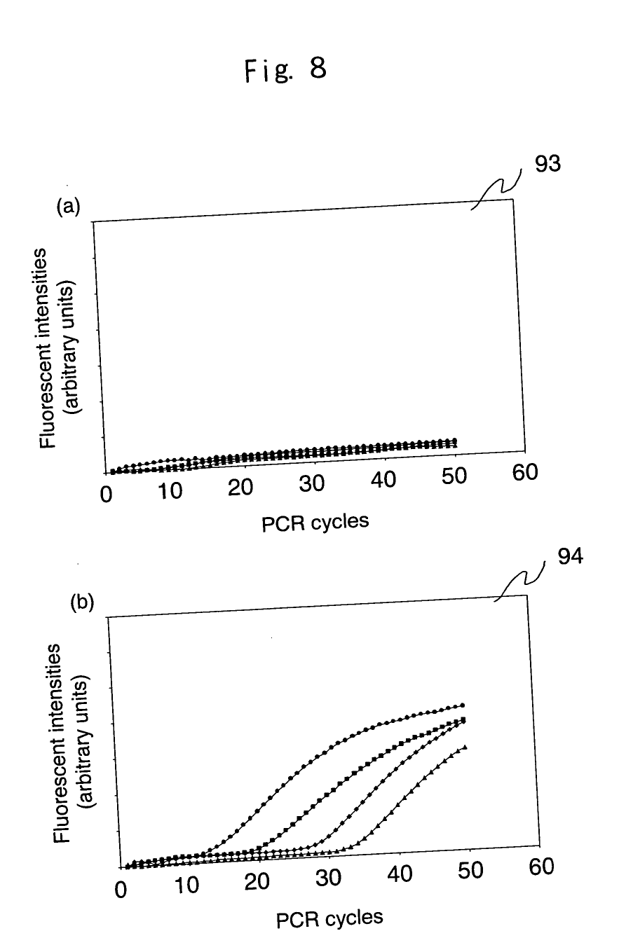

In contrast with FIG. 7, FIG. 8 shows the results of experiments in which a mouse kidney-derived target RNA was used as a template at various concentration levels and two types of probes were used for detection. The horizontal axis of the graph represents PCR cycles, and the vertical axis represents relative fluorescent intensities (arbitrary units) of a fluorophore. FIG. 8(a) is a graph showing changes in the intensity of the fluorescent signal when detecting PCR products of a mouse kidney-derived target RNA using probe A, and FIG. 8(b) is a graph showing that using probe B. As is apparent from FIG. 8(a), a sample did not contain any mouse liver-derived target RNA at all, and thus, a fluorescent signal derived from probe A was not substantially amplified in the PCR cycle. Accordingly, it was confirmed that probe A reacted only with the target RNA derived from a mouse liver, and probe B reacted only with the target RNA derived from a mouse kidney.

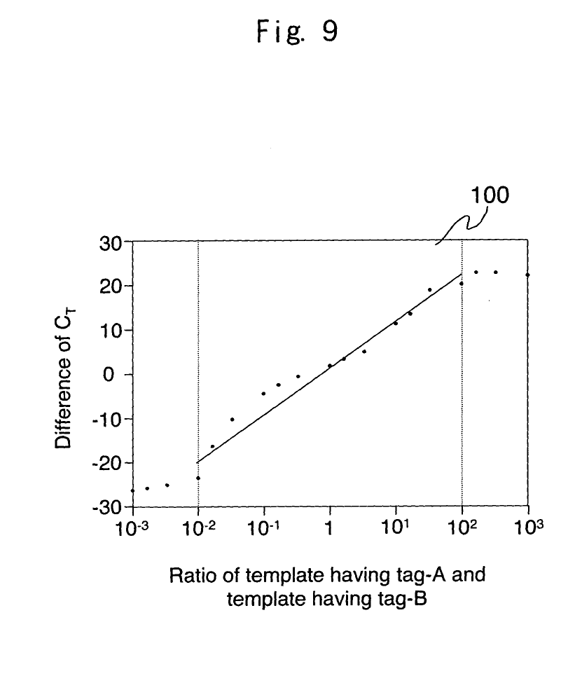

FIG. 9 shows the results of PCR amplification in the same manner using a solution comprising mouse liver-derived target RNA mixed with mouse kidney-derived target RNA at a variety of concentration ratios as templates. When a PCR cycle in which a probe-derived fluorescent signal exceeds the threshold is determined as a threshold cycle (C_{T}), C_{T} is a value that reflects the amount of template (see Dieter K., et al., Electrophoresis, 1999, vol. 20, pp. 291-299). The C_{T} value derived from probe A is determined as C_{TA}, and the C_{T} value derived from probe B is determined as C_{TB}. A difference between C_{TA} and C_{TB} indicates the relative amount of each template. In the graph in FIG. 9, differences between C_{TA} and C_{TB} obtained by the fluorescent signal analysis are plotted. When the ratio of templates (amount of template A/amount of template B) is in the range of 0.01 to 100, the value of C_{TA}-C_{TB} is in a substantially proportional relationship. When the ratio of templates is no more than 0.01 or no less than 100, the graph deviates from the approximated line. Accordingly, it was confirmed that the abundance ratio between two types of templates could be directly compared based on the difference between C_{TA} and C_{TB} when the ratio of templates was within 100-fold or within 1/100.

All publications, patents, and patent applications cited herein are incorporated herein by reference in their entirety.

### Effect of the Invention

The present invention provides a universal probe for gene expression analysis. This probe eliminates the need for repeatedly designing a cost-intensive probe in accordance with the base sequence of a target gene. Any type of target gene can be subjected to real-time PCR detection under substantially the same conditions, and accurate quantitative analysis can be performed. Further, since PCR is carried out in a single thermal cycle, production of by-products can be reduced, and highly accurate quantitative analysis can be performed. Furthermore, competitive PCR using two types of universal probes according to the present invention in one reaction vessel enables simultaneous real-time detection of several specimens. This enables relative comparison of gene expression levels without producing a standard sample.

### Free Text of Sequence Listing

SEQ ID NO: 1—Description of artificial sequence: Reverse transcription primer for synthesizing cDNA from mouse gene GAPDH

SEQ ID NO: 2—Description of artificial sequence: Forward primer for amplifying mouse gene GAPDH

SEQ ID NO: 3—Description of artificial sequence: Reverse primer for amplifying mouse gene GAPDH

SEQ ID NO: 4—Description of artificial sequence: Probe for detecting mouse gene GAPDH with real-time PCR

SEQ ID NO: 5—Description of artificial sequence: Fluorescent-labeled reverse primer for detecting mouse gene GAPDH with real-time PCR

SEQ ID NO: 6—Description of artificial sequence: Reverse transcription primer for synthesizing cDNA from mouse gene GAPDH

SEQ ID NO: 7—Description of artificial sequence: Probe for detecting mouse gene GAPDH with real-time PCR

We claim:

1. A method for gene expression analysis comprising:
subjecting a gene to be analyzed to nucleic acid amplification in a single thermal cycle using a primer comprising a base sequence specifically hybridizing to a target gene, a primer comprising a base sequence identical to a second base sequence, a probe comprising a base sequence identical or complementary to a first base sequence, and labeled at one end with a fluorophore and at another end with a quencher, and thermostable DNA polymerase having 5′→3′ exonuclease activity,
digesting the probe hybridized to the first base sequence by the thermostable DNA polymerase at the time of the nucleic acid amplification, and
detecting a fluorescence emitted by the released fluorophore, thereby assaying the amount of the product of the nucleic acid amplification,
wherein the gene to be analyzed is prepared by introducing the first base sequence and the second base sequence, which are nonspecific to the base sequence of the target gene, into the target gene so that the second base sequence is bound to a position closer to the 5′ end than the first base sequence.

2. The method for gene expression analysis according to claim 1, wherein the gene to be analyzed is synthesized by introducing the first base sequence and the second base sequence into the target gene using a primer for introduction, which comprises the first base sequence, which is closer to the 5′ end than the third base sequence comprising a sequence that specifically hybridizes to the target gene, and the second base sequence, which is closer to the 5′ end than the first base sequence.

3. The method for gene expression analysis according to claim 1, wherein the gene to be analyzed is cDNA comprising the first base sequence and the second base sequence introduced therein by subjecting mRNA of the target gene to reverse transcription using a primer for introduction which comprises the first base sequence, which is closer to the 5′ end than the third base sequence comprising a sequence that specifically hybridizes to the target gene and the second base sequence, which is closer to the 5′ end than the first base sequence.

4. The method for gene expression analysis according to claim 1, wherein samples derived from several specimens are simultaneously analyzed using two or more types of probes using one vessel for one target gene.

5. The method for gene expression analysis according to claim 4, wherein the Tm values of the two or more types of probes are substantially the same.

6. A kit for gene expression analysis comprising:
a probe comprising a sequence identical or complementary to a first base sequence, and labeled at one end with a fluorophore and at another end with a quencher, and
a primer comprising a sequence identical to a second base sequence and bound to a position closer to the 5′ end than the first base sequence,
wherein the first base sequence and the second base sequence are nonspecific to a base sequence of a target gene and are introduced into the target gene.

7. The kit according to claim 6, wherein each of the two or more types of probes comprises several module sequences of 3 or 4 bases, the both terminal bases of each module sequence are identical to each other, and each probe is constituted by rearranging the order of module sequences having the identical terminal bases.


SEQUENCE LISTING (7 sequences)

SEQ ID NO: 1 - DNA, 60 bp, Artificial Sequence
  inventor Uematsu, Chihiro ; Okano Kazunori
tacgactcactctacgcccacccttctcactgttctctcatggatgcagggatgatgttc

SEQ ID NO: 2 - DNA, 19 bp, Artificial Sequence
  Description of Artificial Sequence Forward primer for amplifying mouse gene GAPDH
tgcaccaccaactgcttag

SEQ ID NO: 3 - DNA, 20 bp, Artificial Sequence
  Description of Artificial Sequence Reverse primer for amplifying mouse gene GAPDH
tacgactcactctacgccga

SEQ ID NO: 4 - DNA, 21 bp, Artificial Sequence
  Description of Artificial Sequence Probe for detecting mouse gene GAPDH with real-time PCR
cccttctcactgttctctcat

SEQ ID NO: 5 - DNA, 44 bp, Artificial Sequence
  Description of Artificial Sequence Fluorescent-labeled reverse primer for detecting mouse gene GAPDH with real-time PCR
agcgatgcaccctcagcatcgctcccttctcactgttctctcat

SEQ ID NO: 6 - DNA, 60 bp, Artificial Sequence
  Description of Artificial Sequence Reverse transcription primer for synthesizing cDNA from mouse gene GAPDH
tacgactcactctacgcccacactcatctcttctccctgttggatgcagggatgatgttc

SEQ ID NO: 7 - DNA, 21 bp, Artificial Sequence
  Description of Artificial Sequence Probe for detecting mouse gene GAPDH with real-time PCR
cactcatctcttctccctgtt